(19) United States
(12) Patent Application Publication
(10) Pub. No.: US 2013/0344150 A1
(43) Pub. Date: Dec. 26, 2013

(54) DIRECT COMPRESSION TABLETS OF OTILONIUM

(71) Applicant: Abdi Ibrahim IIac Sanayi Ve Ticaret Anonim Sirketi, Bahcesehir (TR)

(72) Inventors: Farhad FARSHI, Hadimkoy Bahcesehir (TR); Recep AVCI, Hadimkoy Bahcesehir (TR); Urun KANDEMIRER, Hadimkoy Bahcesehir (TR); Serdar SOYLEMEZ, Hadimkoy Bahcesehir (TR); Fikret KOC, Hadimkoy Bahcesehir (TR)

(21) Appl. No.: 13/916,431

(22) Filed: Jun. 12, 2013

Related U.S. Application Data
(63) Continuation of application No. 13/391,988.

(51) Int. Cl. A61K 31/245; A61K 9/28

(52) U.S. Cl. CPC A61K 31/245; A61K 9/28
USPC 424/480; 514/535; 424/474; 424/482; 427/2.14

(57) ABSTRACT

This invention is related to direct compression of otilonium or its pharmaceutically acceptable salt having perfect powder flowability, good tablet weight distribution and no sticking to the punches.

### CROSS-REFERENCE TO RELATED APPLICATION

This application is a continuation of U.S. application Ser. No. 13/391,988, filed Feb. 23, 2012, which is a U.S. National Stage Application of International Application No. PCT/IB2009/053701, filed Aug. 24, 2009, the disclosures of which are incorporated herein in their entireties.

### BACKGROUND OF THE INVENTION

This invention is related to direct compression of otilonium or its pharmaceutically acceptable salt having perfect powder flowability, good tablet weight distribution and no sticking to the dies or punches of tablet press.

Otilonium bromide is used as an antispasmodic for treating spastic painful conditions of the distal section of the intestinal tract, including IBS (irritable bowel syndrome), and is used for the treatment of irritable bowel, pain and spasm of the distal enteric tract. Experimental studies exhibit that otilonium bromide inhibits both baseline and chemically or physically stimulated gastrointestinal-motility. Clinical studies have confirmed otilonium bromide as a spasmolytic drug with a good tolerability profile.

Spasmoctyl® includes otilonium bromide which is on the market as a reference drug.

Spasmoctyl® tablets contain lactose, starch, sodium starch glycolate, magnesium stearate, hypromellose (hydroxypropylmethylcellulose), titanium dioxide and polyethylene glycol.

Spasmoctyl® tablets are prepared by using granulation technics. In the course of preparing Spasmoctyl® tablets, the blended product is granulated prior to pressing (M. Blanco et al., Development and validation of a near infrared method for the analytical control of a pharmaceutical preparation in three steps of the manufacturing process, Fresenius Anal Chem., 2000, 368:534-539, page 535).

Concerning qualitative composition of Spasmoctyl® 40 mg Film Coated Tablets; starch is the only excipient that can be used as a binder. In tablet formulations technology, freshly prepared starch paste at 50°-70° C. is used in tablet granulations as a binder (Handbook of Pharmaceutical Excipients, Fifth Edition, Edited by Raymond C Rowe et al) thus it is understood that in Spasmoctyl® starch is used and it points out that tablet formulation is prepared through using of granulation methods. Meanwhile selection of the quantity should be chosen very carefully because the granulation with starch paste may cause some problems such as poor granule friability, tablet friability, capping, low hardness, disintegration rate, and drug dissolution rate and this kind of manufacturing process with starch paste is much more difficult and requires experienced staff. On the other hand such manufacturing process takes more times and more expenditures.

In pharmaceutical technology, it is well known that if possible, direct compression is preferred rather than granulation. In direct compression, the active and the excipients are blended together and compressed directly without extra processing, such as granulation. Direct compression is the most effective and favorable manufacturing process for the production of solid dosage forms. Thus manufacturers would prefer to use direct compression techniques since it is advantageous overs granulation. Direct compression technique has quick processing and cost advantages.

It is invented that direct compression pharmaceutical compositions of otilonium bromide having eligible granule friability, tablet friability, hardness, disintegration rate, and drug dissolution rate.

### SUMMARY OF THE INVENTION

Thus one of the aims of this invention is to determine direct compression of otilonium. In formulations, a direct compression agent or mixtures of agents are used. Based on this invention, direct compression agents are, but not limited to, pregelatinised starches, polyvinylpyrrolidone, methylcellulose, microcrystalline cellulose, sucrose, lactose, dextrose, sorbitol, mannitol, lactitol, xylitol, modified calcium salt, granulated com starch, modified rice starch, compressible sugars such as Destab™, dextrates such as Emdex® dicalcium phosphate, hydroxypropylcellulose, methylcellulose, hydroxypropylmethylcellulose, polyethylene glycol, amylose, anhydrous calcium hydrogen phosphate, calcium sulphate, tribasic calcium phosphate, dibasic calcium phosphate, low-crystallinity powdered cellulose, silicified-microcrystalline cellulose, chitin, chitosan-hydrochloride, copovidone, croscarmellose sodium, dextrose, anhydrous lactose, anhydrous alpha lactose, anhydrous beta lactose, agglomerated lactose, spray-dried lactose, maltodextrin, mixtures thereof and the like.

Spray dried types of lactose monohydrate are preferred.

Mixtures and/or co-processed diluents which are suitable for direct compression can also be used such as anhydrous lactose-anhydrous lactitol, calcium sulphate-microcrystalline cellulose, lactose-cellulose, lactose-starch, lactose-povidone, Sucrose-maltodextrin coprecipitate and the like.

### BRIEF DESCRIPTION OF THE DRAWING

FIG. 1 is a graph showing dissolution profiles of reference tablets (Spasmoctyl®) and test tablets of the present invention.

### DETAILED DESCRIPTION OF THE PREFERRED EMBODIMENT

According to this invention, the pharmaceutical compositions may further comprise pharmaceutically-acceptable excipient or excipients selected from the group of diluents, disintegrants, binders, lubricants, glidants, mixtures thereof and the like.

When formulating direct compression tablets, the choice of direct compression (DC) binder is extremely critical. It must fulfill certain requirements: good binding functionality and powder flowability are essential. Another functionality of DC binders is their compressibility under pressure. Binders are, but not limited to, polyvinylpyrolidone, copovidone, starches such as pregellatinized starch or plain starch, cellulose derivatives such as hydroxypropylmethylcellulose, ethylcellulose, hydroxypropylcellulose and carboxymethylcellulose and their salts, gelatine, acacia, agar, alginic acid, carbomer, ceratonia, chitosan, dextrates, dextrin, glycerol dibehenate, guar gum, hypromellose, inulin, magnesium aluminum silicate, maltodextrin, poloxamer, polycarbophil, polydextrose, polyethylene oxide, polymethacrylates, sodium alginate, sucrose, hydrogenated vegetable oil, mixtures thereof and the like. Copovidone (vinylpyrrolidone-vinyl-acetate copolymer) is a preferred binder. With copovidone, accelerated and long term stability results did not show any incompatibility at the final product.

The compositions of the present invention preferably also contain a glidant. Glidants are preferably selected from the group consisting of colloidal silicon dioxide, precipitated silica and pyrogenic silica, talc and aluminium silicate, tribasic calcium phosphate, calcium stearate, powdered cellulose, magnesium oxide, magnesium silicate, magnesium trisilicate, starch, talc, mixtures thereof and the like. Colloidal silicon dioxide is preferred. During the development of the product, in order to improve flowability of the final mixture before compression colloidal silicone dioxide is used. Accelerated and long term stability results did not show any incompatibility at the final product.

Disintegrants are, but not limited to, modified starches, croscarmallose sodium, carboxymethylcellulose calcium, sodium starch glycolate, crospovidone, alginic acid, calcium alginate, microcrystalline cellulose, powdered cellulose, chitosan, colloidal silicon dioxide, crospovidone, guar gum, low-substituted hydroxypropyl-cellulose, hydroxypropyl starch, magnesium aluminum silicate, methylcellulose, polacrilin potassium, sodium alginate, starch, pregelatinised starch, mixtures thereof and the like. Sodium starch glycolate is preferred.

Lubricants are, but not limited to, magnesium stearate, calcium stearate, hydrogenated castor oil, glyceryl behenate, glyceryl monostearate, glyceryl palmitostearate, leucine, mineral oil, light mineral oil, myristic acid, palmitic acid, polyethylene glycol, potassium-benzoate, sodium benzoate, sodium lauryl sulfate, sodium stearyl fumarate, stearic acid, talc, hydrogenated vegetable oil, zinc stearate, magnesium lauryl sulphate, sodium stearyl fumarate, polyethylene glycol, stearic acid, colloidal silicon dioxide or mixtures thereof. Preferred lubricant is magnesium stearate.

Diluents are, but not limited to, anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, mannitol, sorbitol, starch, and mixtures thereof.

According to a preferred embodiment of the present invention, the tablet cores are coated. Coating is preferably film coating. Coating agents are, but not limited to, sugars, hydroxypropyl methylcellulose, hydroxypropylcellulose, methylcellulose, ethyl cellulose, polyvinyl alcohol, sodium carboxymethyl cellulose, coatings based on methacrylic acid and its esters, such as Eudragit®, mixtures thereof and the like.

Coating may further contain an excipient or excipients, for example, titanium oxide, talc, ferric oxide, polyethylene glycol and the like. As a coating agent multifunction ingredients such as Opadry®II 85F18422 can also be used.

According to this invention eligible flowability and compressibility is determined with direct compression.

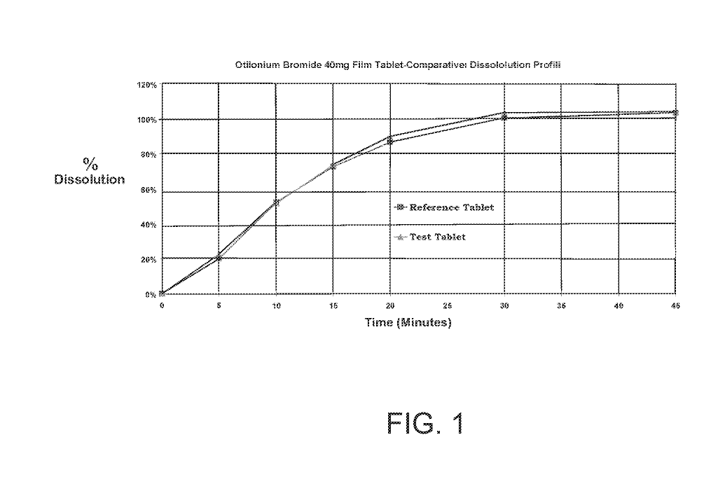

Dissolution profiles of reference tablets (Spasmoctyl®) and test tablets should be same or identical in desired dissolution mediums. Desired dissolution profile means that in targeted dissolution mediums f2 (similarity factor) value should be at least 50 to 100 when compared to the reference Spasmoctyl® dissolution profile. According to this invention direct compression tablets of otilonium (called as test tablet) is in desired ranges of f2 value (FIG. 1).

On the other hand this invention includes a preparation method for direct compression formulations of otilonium. This method comprising steps of a direct compression agent, otilonium bromide and binder are installed into a container and mixed, b. Disintegrant and pre-sieved glidant are installed into container onto powder mixture prepared at step a and mixed, c. Pre-sieved lubricant is installed into container onto powder mixture prepared at step band mixed, d. The final mixture is compressed in a tablet press at 30-70 N average hardness, e. a coating agent or mixtures of coating agents are added into purified water and stirred, f. Core tablets are installed into a coating pan and coated with the coating suspension prepared at step e.

In a embodiment of this invention, diluent is in the range of from about 20% to about 85%, binder is in the range of from about 2% to about 10%, disintegrant is in the range of from about 2% to about 10% , glidant is in the range of from about 0.1% to about 1, lubricant is in the range of from about 0.25% to about 5 by weight of the tablet core.

The following examples are provided to further exemplify the invention and are not intended to limit the scope of the invention.

### EXAMPLE 1

TABLE 1
Formulation Table

| Excipients | % by weight of the tablet core |
|---|---|
| Direct Compression Agent | 50.5 |
| Binder | 5 |
| Disintegrant | 2 |
| Glidant | 0.5 |
| Lubricant | 2 |

1. A Direct compression pharmaceutical composition comprising otilonium or its pharmaceutically acceptable salts and at least one direct compression agent.

2. The direct compression pharmaceutical composition as claimed in claim 1, wherein the direct compression agent is selected from the group consisting of pregelatinised starches, polyvinylpyrrolidone, methylcellulose, microcrystalline cellulose, sucrose, lactose, dextrose, sorbitol, mannitol, lactitol, xylitol, modified calcium salt, granulated com starch, modified rice starch, compressible sugar, dextrate, dicalcium phosphate, hydroxypropylcellulose, methylcellulose, hydroxypropylmethylcellulose, polyethylene glycol, amylose, anhydrous calcium hydrogen phosphate, calcium sulphate, tribasic calcium phosphate, dibasic calcium phosphate, low-crystallinity powdered cellulose, silicified microcrystalline cellulose, chitin, chitosan hydrochloride, copovidone, croscarmellose sodium, dextrose, anhydrous lactose, anhydrous alpha lactose, anhydrous beta lactose, agglomerated lactose, spray-dried lactose, maltodextrin, co-processed anhydrous lactose-anhydrous lactitol, co-processed calcium sulphatemicrocrystalline cellulose, co-processed lactose-cellulose, co-processed lactose-starch, co-processed lactose-povidone, coprecipitated sucrose-maltodextrin, and mixtures thereof.

3. The direct compression pharmaceutical composition as claimed in claim 2, wherein the preferred direct compression agent is spray-dried lactose.

4. The direct compression pharmaceutical composition as claimed in claim 1, wherein the pharmaceutically acceptable salt of otilonium is bromide.

5. The direct compression pharmaceutical composition as claimed in claim 1, further comprising a pharmaceutically acceptable excipient or excipients selected from the group consisting of disintegrants, binders, lubricants, glidants, and mixtures thereof.

6. The direct compression pharmaceutical composition as claimed in claim 5, wherein the glidant is selected from the group consisting of colloidal silicon dioxide, precipitated silica, pyrogenic silica, talc, aluminum silicate, tribasic calcium phosphate, calcium stearate, powdered cellulose, magnesium oxide, magnesium silicate, magnesium trisilicate, starch, talc, and mixtures thereof.

7. The direct compression pharmaceutical composition as claimed in claim 6, wherein the preferred glidant is colloidal silicone dioxide.

8. The direct compression pharmaceutical composition as claimed in claim 5, wherein the disintegrant is selected from the group consisting of modified starches, croscarmallose sodium, carboxymethylcellulose calcium, sodium starch glycolate, crospovidone, alginic acid, calcium alginate, microcrystalline cellulose, powdered cellulose, chitosan, colloidal silicon dioxide, crospovidone, guar gum, low-substituted hydroxypropylcellulose, hydroxypropyl starch, magnesium aluminum silicate, methylcellulose, polacrilin potassium, sodium alginate, starch, pregelatinised starch, and mixtures thereof.

9. The direct compression pharmaceutical composition as claimed in claim 8, wherein the preferred disintegrant is sodium starch glycolate.

10. The direct compression pharmaceutical composition as claimed in claim 5, wherein the lubricant is selected from the group consisting of magnesium stearate, calcium stearate, hydrogenated castor oil, glyceryl behenate, glyceryl monostearate, glyceryl palmi-tostearate, leucine, mineral oil, light mineral oil, myristic acid, palmitic acid, polyethylene glycol, potassium benzoate, sodium benzoate, sodium lauryl sulfate, sodium stearyl fumarate, stearic acid, talc, hydrogenated vegetable oil, zinc stearate, magnesium lauryl sulphate, sodium stearyl fumarate, polyethylene glycol, stearic acid, colloidal silicon dioxide, and mixtures thereof.

11. The direct compression pharmaceutical composition as claimed in claim 10, wherein the referred lubricant is magnesium stearate.

12. The direct compression pharmaceutical composition as claimed in claim 5, wherein the binder is selected from the group consisting of polyvinylpyrolidone, starches, copovidone, hydroxypropylmethylcellulose, ethylcellulose, hydroxypropylcellulose, carboxymethyl-cellulose, gelatine, acacia, agar, alginic acid, carbomer, ceratonia, chitosan, dextrates, dextrin, glycerol dibehenate, guar gum, hypromellose, inulin, magnesium aluminum silicate, maltodextrin, poloxamer, polycarbophil, polydextrose, polyethylene oxide, polymethacrylates, sodium alginate, sucrose, hydrogenated vegetable oil, and mixtures thereof.

13. The direct compression pharmaceutical composition as claimed in claim 12, wherein the preferred binder is copovidone.

14. The direct compression pharmaceutical composition as claimed in claim 5, wherein the excipients of pharmaceutical composition comprising; direct compression agent is in the range of from 20% to 85%, the binder is in the range of from 2% to 10%, the dis-integrant is in the range of from 2% to 10%, the glidant is in the range of from 0.1% to 1, and the lubricant is in the range of from 0.25% to 5 by weight of the tablet core.

15. The direct compression pharmaceutical composition as claimed in claim 1, wherein the pharmaceutical composition is a tablet.

16. The direct compression pharmaceutical composition as claimed in claim 15, wherein the tablet includes a coating.

17. The direct compression pharmaceutical composition as claimed in claim 16, wherein the agents used in coating are selected from the group consisting of sugars, hydroxypropyl methylcellulose, hydroxypropylcellulose, methylcellulose, ethyl cellulose, polyvinyl alcohol , sodium carboxymethyl cellulose, Eudragit®, and mixtures thereof.

18. The direct compression pharmaceutical composition as claimed in claim 15, wherein the tablet is at 30-70 N average hardness.

19. A process for the preparation o f a composition as claimed in claim 1, wherein the comprising the steps of (a) the direct compression agent, the otilonium bromide and the binder are installed into a container and mixed, (b) the dis-integrant and pre-sieved glidant are installed into container onto powder mixture prepared at step a and mixed, (c) the pre-sieved lubricant is installed into container onto powder mixture prepared at step b and mixed, (d) the final mixture is compressed, (e) a coating agent or mixtures of coating agents are added into purified water and stirred, and (f) the tablet cores are installed into a coating pan and coated with the coating suspension prepared at step (e).
